(19) United States
(12) Patent Application Publication
(10) Pub. No.: US 2026/0191961 A1
(43) Pub. Date: Jul. 9, 2026

(54) RUTHENIUM (II) POLYPYRIDYL COMPLEX AS A PHOTO-SENSITISER IN PHOTODYNAMIC THERAPY, AND A PROCEDURE FOR MAKING SAID RUTHENIUM (II) POLYPYRIDYL COMPLEX

(71) Applicants: UNIVERSITÀ DEGLI STUDI DI FIRENZE, Firenze (IT); UNIVERSITÀ DEGLI STUDI DI CAGLIARI, Cagliari (IT)

(72) Inventors: Luca CONTI, Galciana (IT); Gina Elena GIACOMAZZO, Firenze (IT); Claudia GIORGI, Firenze (IT); Barbara VALTANCOLI, Sesto Fiorentino (IT); Alessia DIDDI, Prato (IT); Luca CASULA, Villacidro (IT); Sergio MURGIA, Cagliari (IT); Michele SCHLICH, Cagliari (IT); Chiara SINICO, Capoterra (IT)

(73) Assignees: UNIVERSITÀ DEGLI STUDI DI FIRENZE, Firenze (IT); UNIVERSITÀ DEGLI STUDI DI CAGLIARI, Cagliari (IT)

(21) Appl. No.: 19/131,148

(22) PCT Filed: Nov. 15, 2023
(86) PCT No.: PCT/IB2023/061573

§ 371 Date: May 19, 2025

(30) Foreign Application Priority Data
Nov. 18, 2022 (IT) 102022000023862

(51) Int. Cl. A61K 41/00; C07F 15/00

(52) U.S. Cl. CPC A61K 41/0057; C07F 15/0053

(57) ABSTRACT

A Ruthenium (II) coordination compound: (III) where n is 0 or 2 and R is selected from the group consisting of: methyl; phenyl; —CH₂NH₂; —CH₂OH; —COOH; an ester group —COOR₁, an amide group —CONR₂R₃, for use as photosensitising agents in photodynamic therapy to treat neoplasms. Specifically, a photosensitising medicament comprises compound (III) incorporated into liquid lipid nanoparticles-lamellar and/or non-lamellar crystalline, in particular cubosomes. A procedure to obtain compounds (III) involves three reaction steps (IV).

### SCOPE OF THE INVENTION

The present invention relates to a Ru(II) polypyridyl complex for use as a photosensitiser, i.e. a photosensitising agent, in a photodynamic therapy for treating tumours, to a synthetic procedure thereto and to a medicament including such complex.

### PRIOR ART—TECHNICAL PROBLEMS

In recent years, photodynamic therapy has attracted increasing interest in the scientific community for the treatment of a wide variety of malignancies, as an alternative to chemotherapy and radiotherapy, in order to overcome well-known drawbacks, such as severe side effects, drug resistance and poor drug efficacy.

Photodynamic therapy involves the use of low-energy light-activated photosensitising agents that can be activated to produce highly cytotoxic reactive oxygen species (ROS), including singlet oxygen ¹O₂. Photodynamic therapy has the advantage of allowing a spatio-temporal control over the activation of the photosensitizer, permitting to improve the selectivity and efficacy of the treatment against selected tumoral targets, besides containing the side effects commonly associated with non-specific drug delivery.

A photosensitizer for photodynamic therapy must possess low toxicity under dark conditions and a high capacity to produce ROS after photoexcitation. Most to date known photosensitizers comprise tetrapyrrolic structures, such as porphyrins, phthalocyanines and chlorines. These drugs, despite their high capacity to produce cytotoxic species after irradiation, are poorly soluble in biological media, poorly selective, and induce prolonged photosensitivity in patients.

Some transition metal complexes, employed as photosensitising agents in photodynamic therapy, are likely to overcome these drawbacks. In particular, polypyridyl Ru(II) complexes show advantageous photophysical and electrochemical properties including high thermodynamic and kinetic stability, as well as the capability to generate singlet oxygen with high quantum yields. Examples of such compounds can be found in WO2020260424A1, EP3521295, CN113321687A, CN111875643A, CN110857310A, CN112266402A and WO2021032952A1.

In recent years, several Ru(II) polypyridyl complexes containing a benzo[i]dipyrido[3,2-a:2′,3′-c]phenazine unit, hereafter referred to as ‘dppn’, have been synthesized. This ligand includes a particularly extensive aromatic system, which makes it possible to adjust the chemical-physical properties of the obtained compounds in a wide range, in particular, it allows to maximize the yield of singlet oxygen production, from which excellent therapeutic outcomes are derived.

A very limited number of polypyridyl complexes comprising two dppn units have also been synthesized. Two publications are known in this respect, which are mentioned below.

In a paper by Wang and co-workers¹, reference is made to a compound of formula:

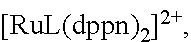

where L is 3,8-di(benzothiazolylfluorenyl)-1,10-phenanthroline, obtained by first introducing the two dppn units and then reacting the ligand L with the intermediate [Ru(dppn)₂Cl₂] in ethylene glycol. However, details on the way to carry out the synthesis of this intermediate are omitted and essential characterization data, such as ¹H NMR spectra of [RuL(dppn)₂]²⁺, were not provided. Such a synthetic route, which occurs via the intermediate [Ru(dppn)₂Cl₂], has the drawback that this intermediate is poorly soluble in most organic solvents, resulting in complications in the inherent synthesis.

A paper by Turro and co-workers² refers to the synthesis and characterization of a compound with formula:

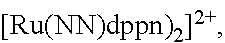

where NN is ‘bpy’=2,2′-bipyridine. In this case, the preparation of the metal complex was carried out by the preliminary synthesis of the intermediate [Ru(NN)Cl₄] and then [Ru(NN)(CH₃CN)₄]²⁺, avoiding the use of [Ru(dppn)₂Cl₂], and introducing the two dppn groups only in the subsequent reaction steps. However, reaction times from 7 to 24 days were required to obtain [Ru(NN)Cl₄], which are very time-consuming and, therefore, industrially unacceptable. A number of complex purification steps are also required by this approach, leading to relatively low overall yields, of 28-37%. It is worthwhile to note that, in the above cited report by Turro, the compound [Ru(NN)(dppn)₂]²⁺ has only been studied as dye for solar cell applications (DSSC).

It is therefore desirable to define a route for the synthesis of polypyridyl Ru(II) complexes containing two dppn units that does not have the drawbacks of the prior art techniques briefly discussed above, i.e. a route providing intermediate compounds that are easily soluble in organic solvents and that allows the desired complexes to be obtained at higher yields and in shorter reaction times.

### SUMMARY OF THE INVENTION

It is therefore an object of the present invention to provide a class of coordination compound or Ru(II) complexes that are suitable for the use as photosensitising agents in the photodynamic therapy of neoplasms.

It is another particular object of the invention to provide such a class of Ru(II) complexes that exhibit a greater selectivity towards tumour cells than the photosensitising agents for photodynamic therapy that are currently in use.

It is also a particular object of the invention to provide such a class of Ru(II) complexes that are capable of maximising the singlet oxygen production, thereby exhibiting better therapeutic efficiency than the photosensitising agents for photodynamic therapy that are currently in use.

It is another object of the present invention to provide an innovative and straightforward synthetic process for such a class of Ru(II) complexes that allows to obtain the above-mentioned complexes with higher yields and shorter reaction times, if compared to prior art procedures.

It is a further object of the invention to provide a medicament including such a Ru(II) complex, in which two dppn (benzo[i]dipyrido[3,2-a:2′,3′-c]phenazine) ligands are present, while being adequately soluble in water and therefore in biological media, so as to improve absorption by a patient, and distribution to a target site in the patient's body without using potentially harmful cosolvents.

The above-mentioned objects are achieved by ruthenium (II) complexes as disclosed in claim 1, by a process for making said complexes as disclosed in claim 8, and by a photosensitizer medicament for photodynamic therapy as disclosed in claim 4. Advantageous embodiments of complexes and the medicament, as well as advantageous modification of the process, are defined in the respectively dependent claims.

According to one aspect of the invention, a class of ruthenium (II) coordination compounds having the following formula is described:

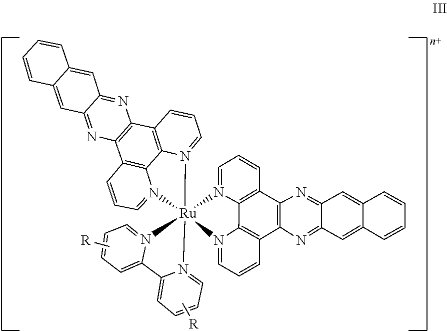

wherein n is selected between 0 and 2,
wherein R are substituent groups selected from the group consisting of:
   a methyl group;
   a phenyl group;
   a —CH₂NH₂ group;
   a —CH₂OH group;
   a carboxyl group —COOH;
   an ester group of formula —COOR₁, where R₁ can be selected from the group consisting of:
      a linear or branched alkyl group with a number of carbon atoms in the range from 1 to 4;
      an unsubstituted benzyl group;
      a polyethylene glycol methoxy group with formula H(OCH₂CH₂)₁₋₄OH;
   an amide group of formula —CONR₂R₃,
   where
      R₂ can be hydrogen and R₃ can be a group selected from the group consisting of:
         a linear or branched alkyl group with a number of carbon atoms in the range from 1 to 4;
         a —(CH₂)₂NH₂ group;
         a phenyl group,
   or
      R₂ and R₃ can be linear or branched alkyl groups with a number of carbon atoms in the range from 1 to 4,
   or
      NR₂R₃ can be a group selected from the group consisting of:
         a cycloalkyl secondary amine with a number of carbon atoms in the range from 4 to 5;
         a heterocycloalkyl secondary amine comprising a heteroatom selected between nitrogen and oxygen,
            for use as a photosensitising agents in the photodynamic therapy of neoplasms.

In other words, the invention relates to a plurality of polypyridyl complexes of ruthenium (II), hereinafter also referred to as [Ru(NN)(dppn)₂]ⁿ⁺, where
dppn are benzo[i]di-pyrido[3,2-a:2′,3′-c]phenazine units, i.e. bidentate heterocyclic ligands characterized by extensive aromaticity, and
NN is a functionalised 2,2′-bipyridine ligand, whose R groups are described above.

The presence of the two dppn ligands in the same molecule makes it possible to maximize singlet oxygen production, therefore the above ruthenium (II) compounds turn out to be useful as photosensitising agents in a photodynamic therapy. More in detail, dppn ligands exhibit extensive π-conjugation, which allows them to easily interact with important biological targets such as proteins and/or DNA, in the latter case the interactions are π-stacking interactions with the DNA double helix. Moreover, the extensive π-conjugation of dppn prolongs the duration of the lowest-energy triplet excited states of the photosensitising agents, leading to improved production of singlet oxygen ¹O₂ due to excitation with appropriate low-energy light radiation, a key feature for the effectiveness of PS potentials.

In fact, as shown hereinafter, the obtained Ru(II) compounds exhibit excellent singlet oxygen production properties via photoexcitation, effectively interact with DNA as a possible biological target, and show remarkable phototoxicity against various cancer cell models.

According to another aspect of the invention, a process for obtaining a compound of a class of ruthenium (II) coordination compounds having the following formula:

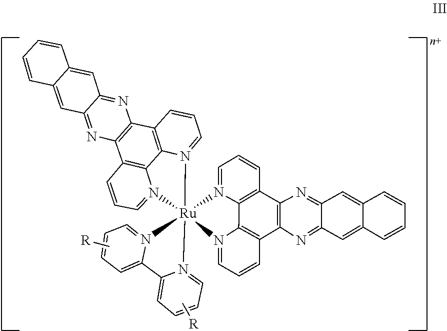

wherein n is selected between 0 and 2,
wherein R are substituent groups selected from the group consisting of:
   hydrogen;
   a methyl group,
   a phenyl group,
   a —CH₂NH₂ group,
   a —CH₂OH group
   a carboxyl group —COOH,
   an ester group of formula —COOR₁, where R₁ can be selected from the group consisting of:
      a linear or branched alkyl group with a number of carbon atoms in the range from 1 to 4;
      an unsubstituted benzyl group;
      a polyethylene glycol methoxy group with formula H(OCH₂CH₂)₁₋₄OH.
   an amide group of formula —CONR₂R₃,
   where
      R₂ can be hydrogen and R₃ can be a group selected from the group consisting of:
         a linear or branched alkyl group with a number of carbon atoms in the range from 1 to 4;
         a —(CH₂)₂NH₂ group;
         a phenyl group,
   or
      R₂ and R₃ can be linear or branched alkyl groups with a number of carbon atoms in the range from 1 to 4,
   or
      NR₂R₃ can be a group selected from the group consisting of:
         a cycloalkyl secondary amine with a number of carbon atoms in the range from 4 to 5;
         a heterocycloalkyl secondary amine comprising a heteroatom selected between nitrogen and oxygen,
comprises the consecutive steps of:
   obtaining a reactive polymeric precursor [Ru(CO)₂Cl₂]ₘ by reaction between ruthenium (Ill) chloride and paraformaldehyde;
   obtaining a trans-Cl[Ru(NN)Cl₂(CO)₂] intermediate complex:

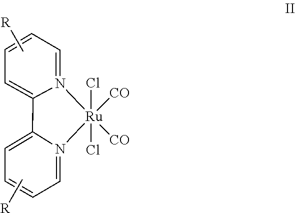

where NN is a functionalised 2,2′-bipyridine ligand, whose R groups are described above,
by reaction of the reactive polymer precursor [Ru(CO)₂Cl₂]ₘ with a predetermined symmetrically functionalised 2,2′-bipyridine ligand with two equal R functional groups;
obtaining the ruthenium (II) coordination compound by reaction of the intermediate complex

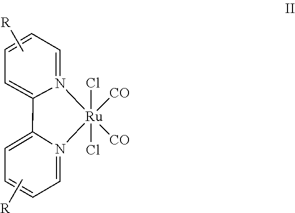

with benzo[i]dipyridine[3,2-a:2′,3′-c]phenazine.

In other words, the present invention provides a straightforward synthetic route to heteroleptic ruthenium (II) complexes that include two extended aromatic bidentate ligands containing nitrogen atoms consisting in the benzo[i]dipyridine[3,2-a:2′,3′-c]phenazine (dppn) units plus a symmetrically functionalised 2,2′-bipyridine ligand whose R-groups are indicated hereinafter, according to the general scheme below,
where complexes of formula III are represented without their charge, and in which three reaction steps are involved, i.e.:

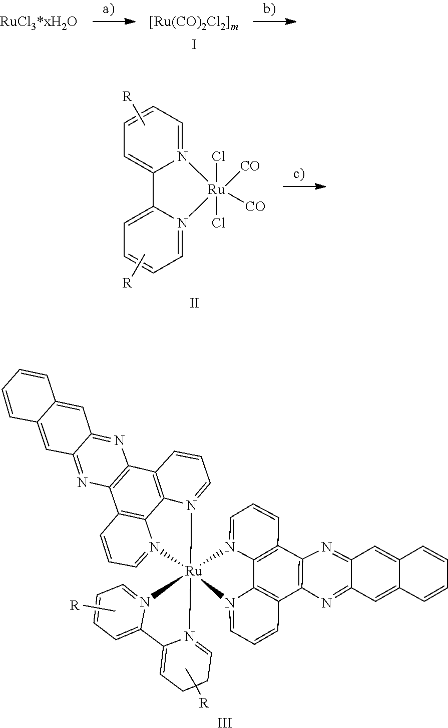

a) preparation of the polymeric precursor I;
b) preparation of trans-Cl[Ru(NN)Cl₂(CO)₂] complexes of formula II;
c) preparation of complexes of formula [Ru(NN)(dppn)₂]ⁿ⁺, with n=0.2 (III) by reaction with two dppn equivalents.

This three-step synthetic procedure allows the desired compounds to be obtained with relatively high yields, normally between 22% and 47% on the overall reaction yields, and the reaction times are as long as a few hours at most.

In the process according to the invention, the NN ligands is introduced before the Ru(II)-coordination by the dppn ligands. In the present synthetic process, complexes of formula III are obtained through the preliminary synthesis of intermediates II, overcoming the problems associated to the scarce solubility in most organic solvents of the intermediate [Ru(dppn)₂Cl₂], conventionally employed in the synthesis of bis-heteroleptic Ru(II) polypyridyl complexes, see the above referenced document by Wang, cit. Typically, this is also the strategy commonly employed to prepare Ru(II) complexes including only one dppn unit.

On the other hand, the process of the present invention allows obtaining the Ru(II) complexes employing a reaction time far shorter than the time required by the route according to Turro et al. (cit.)

The present invention provides a simple method for preparing a complex of the class of complexes [Ru(NN)(dppn)₂]ⁿ⁺ (n=0, 2) with relatively high yields, relying on the preliminary preparation of the reactive polymeric compound [Ru(CO)₂Cl₂]ₘ (I), which is first reacted with the functionalized 2,2′-bipyridine unit to give the monomer trans-Cl[Ru(NN)Cl₂(CO)₂](II), which is then allowed to react with two equivalents of dppn units to give compounds Ill. This approach makes it possible to overcome the solubility issues arising from the use of the intermediate [Ru(dppn)₂Cl₂], and allows obtaining variously substituted Ill compounds with good yields.

Moreover, with the synthetic route described here, reaction intermediates I and II can be used without requiring purification, which shortens the overall process times, reduces the required solvent amount and increases the yield. Only compounds Ill require purification before they can be handled for characterisation and further use.

The R-functions of the remaining bidentate NN ligands can be optimally selected to finely tune the chemical-physical properties of the obtained Ru(II) complexes.

Preferably, the 2,2′-bipyridine ligands are functionalized in the 5,5′ and 4,4′ positions with R substituents. In other words, each of said R substituent groups is bonded to a carbon atom at a position selected between the 5,5′ position and the 4,4′ position of each pyridine ring of the 2,2′-bipyridine group

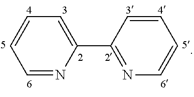 [SMILES: c1ccc(-c2ccccn2)nc1]

The positions indicated above are in fact those that make it possible to obtain compounds that are stable upon irradiation as they are free from such unfavourable conditions as steric encumbrance, and that, at the same time, do not entail complications when synthesising the complex.

In particular, the 2,2′-bipyridine ligand is symmetrically modified in the 5,5′ or 4,4′ positions with two R-substituents. In other words, the substituent groups R are bonded to carbon atoms at symmetrical 5,5′ or 4,4′ positions of respective pyridine rings of this 2,2′-bipyridine group. This makes it possible to obtain products as enantiomeric mixtures avoiding the formation of constitutional isomers.

According to another aspect of the invention, a photosensitising medicament for use in a photodynamic therapy for treating neoplasms is also provided, said medicament containing, as a photosensitising agent, a ruthenium (II) coordination compound having the formula:

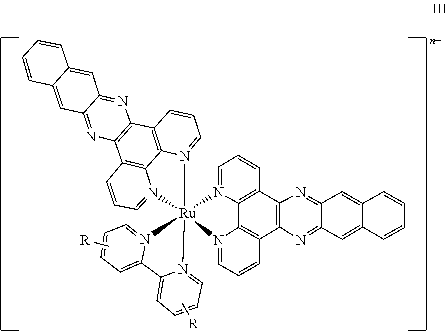

wherein n is selected between 0 and 2,
wherein R are substituent groups selected from the group consisting of:
   hydrogen,
   a methyl group,
   a phenyl group,
   a —CH₂NH₂ group,
   a —CH₂OH group
   a carboxyl group —COOH,
   an ester group of formula —COOR₁, where R₁ can be selected from the group consisting of:
      a linear or branched alkyl group with a number of carbon atoms in the range from 1 to 4;
      an unsubstituted benzyl group;
      a polyethylene glycol methoxy group with formula H(OCH₂CH₂)₁₋₄OH.
   an amide group of formula —CONR₂R₃, where
      R₂ can be hydrogen and R₃ can be a group selected from the group consisting of:
         a linear or branched alkyl group with a number of carbon atoms in the range from 1 to 4;
         a —(CH₂)₂NH₂ group;
         a phenyl group,
   or
      R₂ and R₃ can be linear or branched alkyl groups with a number of carbon atoms in the range from 1 to 4,
   or
      NR₂R₃ can be a group selected from the group consisting of:
         a cycloalkyl secondary amine with a number of carbon atoms in the range from 4 to 5;
         a heterocycloalkyl secondary amine comprising a heteroatom selected between nitrogen and oxygen.
            wherein the coordination compound is incorporated into nanoparticles, in particular, it is incorporated in liquid-crystalline lamellar and/or non-lamellar liquid-crystalline lipid nanoparticles. The effect of such incorporation consists in improving water solubility of the coordination compound and, therefore, in allowing its administration without using any co-solvent.

In other words, the invention relates to a plurality of polypyridyl ruthenium (II) complexes, hereinafter also referred to as [Ru(NN)(dppn)₂]ⁿ⁺, wherein, in each of said complexes,
dppn are benzo[i]di-pyrido[3,2-a:2′,3′-c]phenazine units, i.e. bidentate heterocyclic ligands characterised by extensive aromaticity, and
NN is a functionalized 2,2′-bipyridine ligand, wherein the R groups are as described above.

This allows for one or more therapeutic advantages among:
a) more uniform and/or more selective medicament distribution at the tumour site;
b) crossing epithelial and organ barriers, depending on the route of administration. For example, such barriers may be the stratum corneum of the skin, blood-brain barrier, oesophageal or intestinal mucosa and others;
c) modulation of release and half-life in circulation;
d) reduction of toxicity at non-target sites.

Preferably, the liquid-crystalline lipid nanoparticles are cubosomes.

Cubosomes, often referred to as “bicontinuous cubic liquid crystalline nanoparticles”, are peculiar nanoparticles used for the delivery of lipophilic and/or hydrophilic drugs. They are characterized by a three-dimensional, honeycomb-like arrangement of a lipid bilayer, with inner aqueous channels, and can encapsulate numerous bioactive components, such as pharmacological agents, proteins and amino acids. Generally, cubosomes exhibit several benefits over other nanoparticles, in particular, liposomes, taken as golden standard in nanomedicine, in terms of:
stability: from a colloidal perspective, cubosomes exhibit a greater breaking resistance, a quality that is critical for drug delivery as it increases the probability that the medication will reach the target location without deteriorating or losing its effectiveness;
increased drug loading: cubosomes exhibit a larger bilayer area to particle volume ratio, and they show a huge internal surface area that allows carrying of large pharmacological payloads;
improved bioavailability: by overcoming solubility and stability drawbacks, cubosomes can increase drugs bioavailability, which allows a lower drug dose, thus reducing the possibility of adverse effects.

As a result of the above, cubosomes have shown a higher encapsulation efficiency and a more potent pharmacological effect, both in vitro and in vivo, in comparison e.g. with liposomes.

Similarly, cubosomes showed higher in vitro cytotoxicity, cellular uptake and tumor growth inhibition when compared to solid lipid nanoparticles (SLNs) loaded with a same drug. Moreover, when applied on the skin, cubosomes have shown higher drug penetration compared to liposomes, transfersomes, and ethosomes.

As an alternative, the liquid-crystalline lipid nanoparticles can be selected from the group consisting of:
hexosomes;
solid lipid nanoparticles;
nanoemulsions;
a combination of the above.
   As a further alternative, the liquid-crystalline lipid nanoparticles could also be liposomes.

It also falls within the scope of the invention a photodynamic therapeutic method for treating neoplasms comprising a step of administering to a patient a medicament containing the nanoformulated coordination compound described above as a photosensitising agent, and a step of administering light to a neoplastic area in order to trigger the singlet oxygen production and induce site-specific cytotoxicity.

### BRIEF DESCRIPTION OF THE DRAWINGS

Further features and advantages of the present invention will be better understood by the following description of variants and embodiments thereof, made by way of example and not of limitation, with reference to the attached figures, in which

FIG. 1 shows the ¹H-NMR spectrum of complex IIIa in (CD₃)₂CO);

FIG. 2 shows the ¹H ¹H-COSY spectrum of complex IIIa in (CD₃)₂CO);

FIG. 3 shows the ¹³C-NMR spectrum of complex IIIa in (CD₃)₂CO;

FIG. 4 shows the HSQC spectrum of complex IIIa in (CD₃)₂CO);

FIG. 5 shows the HR MS spectrum of complex IIIa;

FIG. 6 shows the ¹H-NMR spectrum of complex IIIb in (CD₃)₂SO;

FIG. 7 shows the ¹H¹H-COSY spectrum of complex IIIb in (CD₃)₂SO;

FIG. 8 shows the HR MS spectrum of complex IIIb;

FIG. 9 shows the ¹H-NMR spectrum of complex IIIc in (CD₃)₂CO;

FIG. 10 shows the ¹H¹H-COSY spectrum of complex IIIc in (CD₃)₂CO;

FIG. 11 shows the ¹³C NMR spectrum of complex IIIc in (CD₃)₂CO;

FIG. 12 shows the HSQC spectrum of complex IIIc in (CD₃)₂CO;

FIG. 13 shows the HR MS spectrum of complex IIIc;

FIG. 14 shows the absorption spectra of complexes IIIa, IIIb and IIIc in CH₃CN;

FIG. 15 shows the absorption spectra of a solution containing DHN (330 μM) and the complexes IIIa, IIIb and IIIc (10 μM) in acetonitrile for different irradiation times, up to 200 seconds, with a 30 W LED lamp, and an emission spectrum width ranging from 400 nm to 500 nm;

FIG. 16 shows the semilogarithmic graphs of In(Aₜ/Aₒ) as a function of an irradiation time for the complexes IIIa, IIIb and IIIc and for [Ru(phen)₃]Cl₂, the latter taken as a reference ([DHN]=330 μM, [Ru]=10 μM);

FIG. 17 shows the absorption spectra of buffered Tris-HCl 10 mM, NaCl 50 mM solutions at pH 7.2 of complexes IIIa, IIIb and IIIc (10 μM), said spectra detected in the presence of increasing concentrations of double-stranded DNA;

FIG. 18 shows the graphs of the [DNA]/|εₐ-ε_{b}| values against the molar concentration of DNA for the complexes IIIa, IIIb and IIIc, Ru=10 μM, Tris-HCl buffer 10 mM, NaCl 50 mM, pH 7.2;

FIG. 19 shows the results of cytotoxicity tests of complex IIIa, at different concentrations thereof, on squamous cell carcinoma of the epidermis (A431) and glioblastoma (U87MG) models, after irradiation with LED light for 30 minutes and in the dark;

FIG. 20 shows the results of cytotoxicity tests of complex IIIb, at different concentrations thereof, on the squamous cell carcinoma model of the epidermis (A431), after irradiation with LED light for 30 minutes and in the dark;

FIG. 21 shows the results of cytotoxicity tests of complex IIIb encapsulated in cubosomes, at different concentrations, on the squamous cell carcinoma model of the epidermis (A431) after irradiation with LED light for 30 minutes and in the dark;

FIG. 22 shows the results of cytotoxicity tests of complex IIIc, at different concentrations thereof, on the Methastatic Lung Adenocarcinoma (Calu-3), after irradiation with LED light for 30 minutes and in the dark;

FIG. 23 shows the results of cytotoxicity tests of complex IIIc encapsulated in cubosomes, at different concentrations, on the Methastatic Lung Adenocarcinoma (Calu-3) after irradiation with LED light for 30 minutes and in the dark;

FIG. 24 shows a transmission electron microscopy image at cryogenic temperatures of cubosomes loaded with the complex IIIb;

FIG. 25 shows a SAXS diffractogram of cubosomes loaded with complex IIIb, in which the Bragg peaks corresponding to the bicontinuous cubic phases Im3m and Pn3m are indicated by the dashed and continuous vertical lines, respectively;

FIG. 26 shows the results of stability tests of the cubosome formulation.

### EXAMPLES

Three examples of Ru(II) complexes containing two benzo[i]dipyrido[3,2-a:2′,3′-c]phenazine (dppn) units, are described hereinafter. These exemplary complexes can be represented by the common formula:

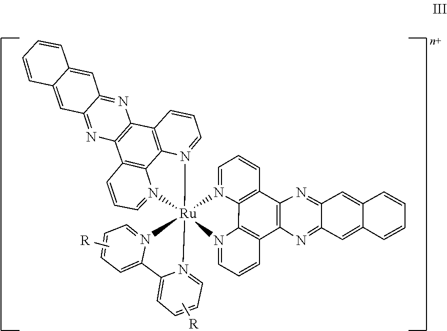

and are denoted IIIa, IIIb and IIIc, according to whether R is a methyl group, a carboxylate group and a morpholinomethanone group, respectively. Therefore, complexes IIIa, IIIb and IIIc have the formulae shown below, in which the charge n+ is omitted:

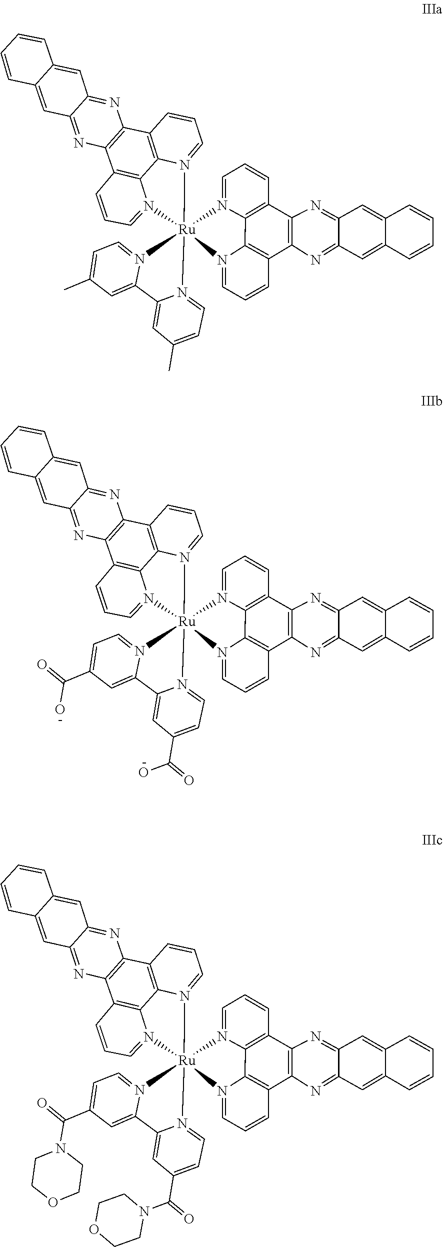

A synthetic route to obtain the above complexes, according to the process of the present invention, is diagrammatically shown below, including steps a), b) and c) leading to intermediates I and II, and to the desired Ru(II) complexes (III), respectively:

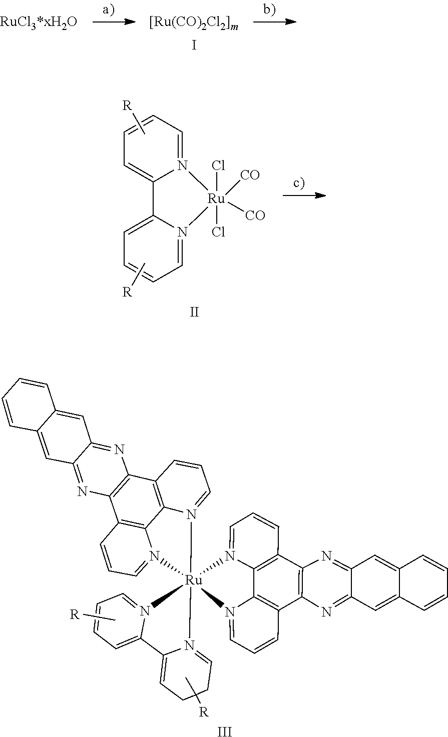

Even in this case, the charge of complex III is omitted for the sake of simplicity.

### Synthesis

### Step (a): Preparation of the Polymer Precursor ‘I’

This common intermediate was prepared in all three cases by refluxing commercial ruthenium trichloride, RuCl₃·xH₂O, and paraformaldehyde in a 1.4:1 w/w in 90% formic acid during 6 hours. The resulting polymer I was isolated as a pale-yellow powder through trituration from hexane. The polymer I precursor was obtained at an 80% yield.

### (b) Preparation of the trans-CI[Ru(NN)Cl₂(CO)₂] Intermediates, ‘II’

The NN ligands corresponding to complexes IIIa, IIIb and IIIc were inserted into the coordination sphere of the Ru(II) centres, leading to the respective intermediate complexes II trans-Cl[Ru(NN)Cl₂(CO)₂]. The polymer precursor I was reacted with the NN ligands in a 1:1 molar ratio in hot methanol or dimethylformamide, depending on the solubility of the specific NN ligand employed. Intermediate complexes II were obtained by a simple hot filtration of the reaction mixture, with high yields, ranging between 55% and 75%.

### (c) Preparation of the Complexes ‘IIIa-IIIc’ from Corresponding Intermediates II

In the third step of the synthetic route to complexes IIIa, IIIb and IIIc, two equivalents of dppn are allowed to react in each case with the respective precursor II in refluxed 2-methoxyethanol as a solvent in the presence of 5 equivalents of trimethylamine N-oxide, leading to the formation of the respective complexes III of formula [Ru(NN)(dppn)₂]ⁿ⁺ (n=0, 2). The complexes are then precipitated by adding a 0.1 M solution of KPF₆, filtered under vacuum and collected with a high degree of purity. When necessary, the complexes are subsequently purified by flash chromatography on silica gel, using a dichloromethane and methanol gradient mixture as the eluent. The desired products are therefore obtained as hexafluorophosphate salts [Ru(NN)(dppn)₂](PF₆)₂ with yields ranging between 50% and 78%.

A detailed description of step (c) of the synthesis of each complex IIIa, IIIb and IIIc is given hereinafter.

### (Ru(4,4′-dimethyl-2,2′-bipyridine)(benzo[i]dipyrido[3,2-a:2′,3′-c]phenazine)₂[PF₆]₂

To a solution of trans-CI[Ru(4,4′-dimethyl-2,2′-bipyridine)Cl₂(CO)₂](80 mg, 0.19 mmol) in 8 mL of degassed 2-methoxyethanol were added benzo[i]dipyrido [3,2-a:2′,3′-c]phenazine (dppn) (129 mg, 0.39 mmol) and trimethylamine N-oxide (106 mg, 0.95 mmol). The reaction mixture was stirred for 4 h at reflux under nitrogen atmosphere. After cooling to room temperature, the addition of 3 mL of 0.1 M KPF₆ solution allowed the complete precipitation of IIIa. The precipitate was filtered and washed with water and chloroform. The crude product was purified by flash chromatography on silica gel (eluent: starting from DCM:MeOH 50:1 with 10% Acetone to DCM:MeOH 30:1 with 10% Acetone) to obtain IIIa as a red powder at a 78% yield.

### [Ru(4,4′-dicarboxylate-2,2′-bipyridine)(benzo[i]dipyrido[3,2-a:2′,3′-c]phenazine)₂] (IIIb)

To a solution of trans-CI[Ru(4,4′-dicarboxylate-2,2′-bipyridine)Cl₂(CO)₂](80 mg, 0.17 mmol) in 8 mL of degassed 2-methoxyethanol were added benzo[i]dipyrido[3,2-a:2′,3′-c]phenazine (dppn) (113 mg, 0.34 mmol) and trimethylamine N-oxide (95 mg, 0.85 mmol). The reaction mixture was stirred during 6 hours at reflux under nitrogen atmosphere. After cooling to room temperature, the crude product was filtered and washed with water and then triturated with CHCl₃ to obtain (IIIb) as a red powder with a 50% yield.

### [Ru(4,4′-(diylbis(morpholinomethanone)-2,2′-bipyridine)(benzo[i]dipyrido[3,2-a:2′,3′-c]phenazine)₂][PF₆]₂ (IIIc)

To a solution of trans-Cl[Ru(4,4′-(diylbis(morpholinomethanone)-2,2′-bipyridine)Cl₂(CO)₂](100 mg, 0.16 mmol) in 8 mL of degassed 2-methoxyethanol was added benzo[i]dipyrido[3,2-a:2′,3′-c]phenazine (dppn) (106 mg, 0.32 mmol) and trimethylamine N-oxide (116 mg, 1.04 mmol). The reaction mixture was stirred during 4 h at reflux under N atmosphere₂. After cooling to room temperature, the addition of 2 mL of 0.1 M aqueous solution of KPF₆ allowed the complete precipitation of complex IIIc. The crude product was filtered and washed with water, methanol and diethyl ether. Subsequently, it was purified by flash chromatography on silica gel (eluent: starting with DCM:MeOH 30:1 with 10% acetone) to obtain complex IIIc as a red solid with a yield of 62%.

The three-step synthesis process resulted in final products with yields ranging from 22% to 47%. The products were obtained pure and characterized by nuclear magnetic resonance ¹H, ¹H¹H COSY, ¹³C NMR and HR MS.

### Characterization of Synthesized Complexes: NMR

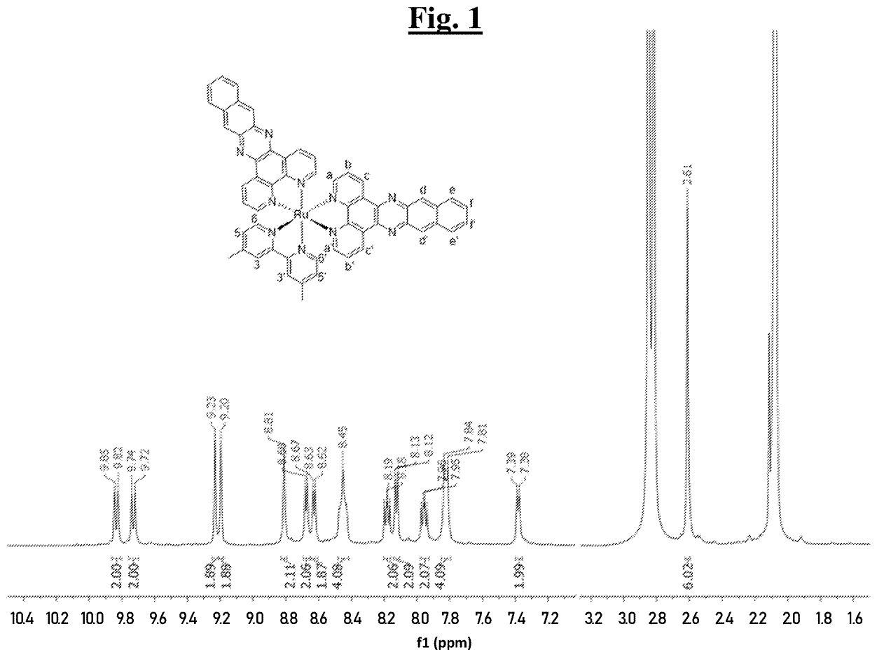

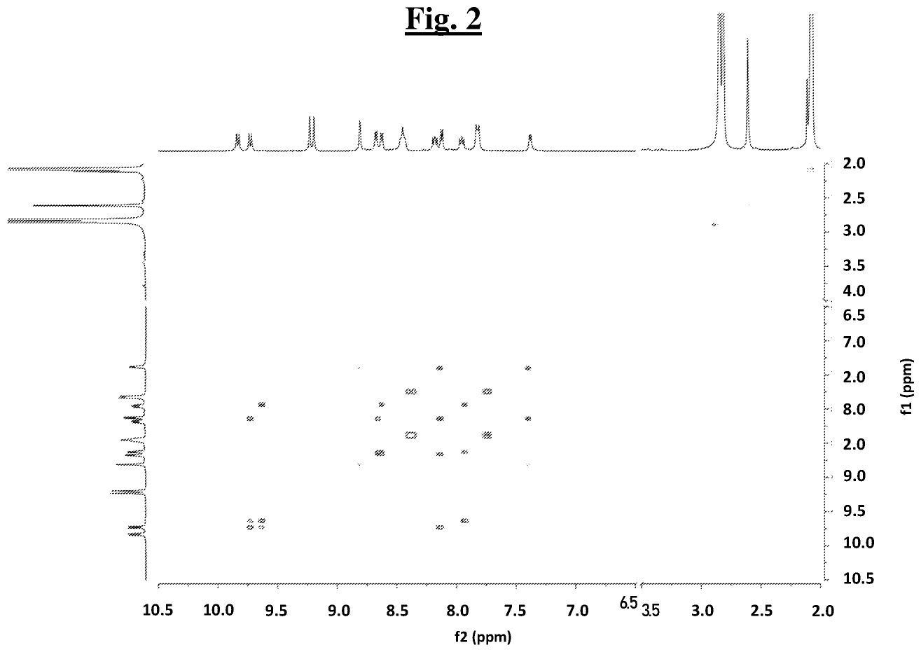

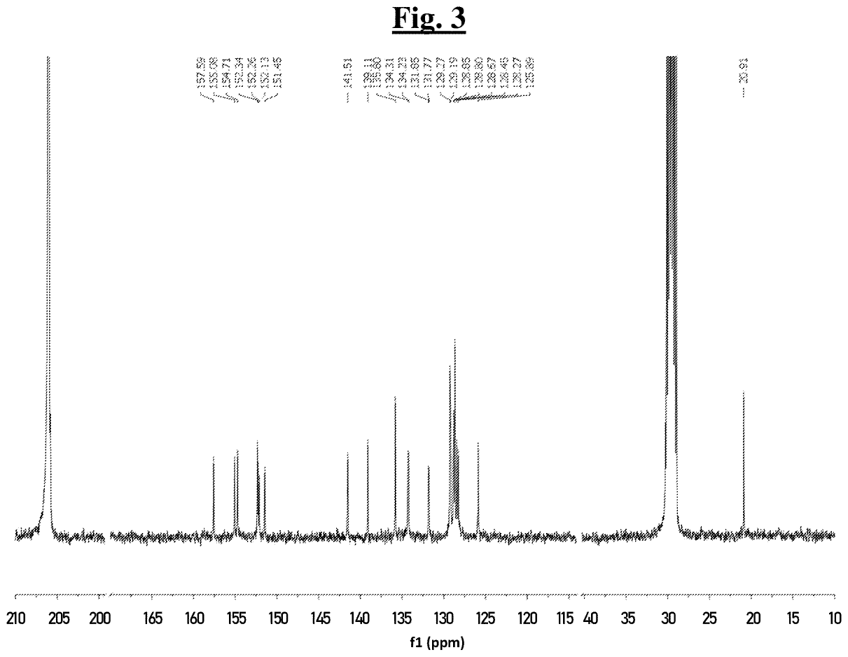

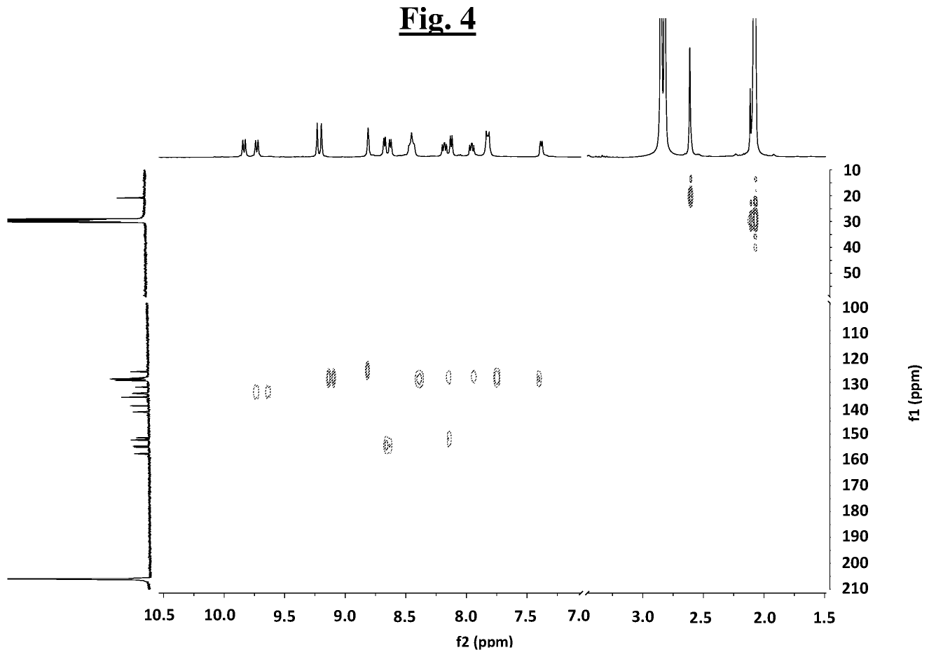

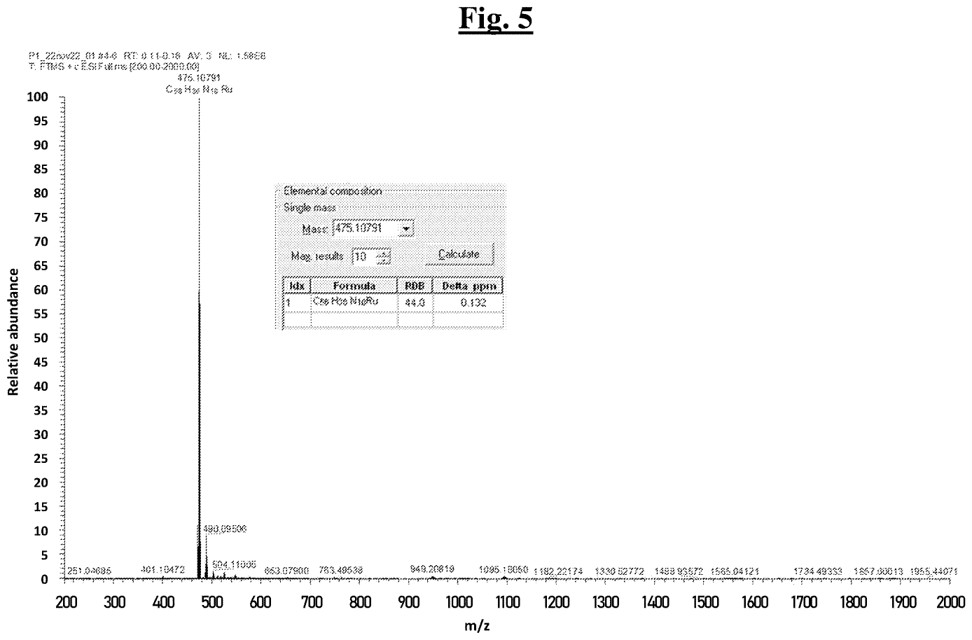

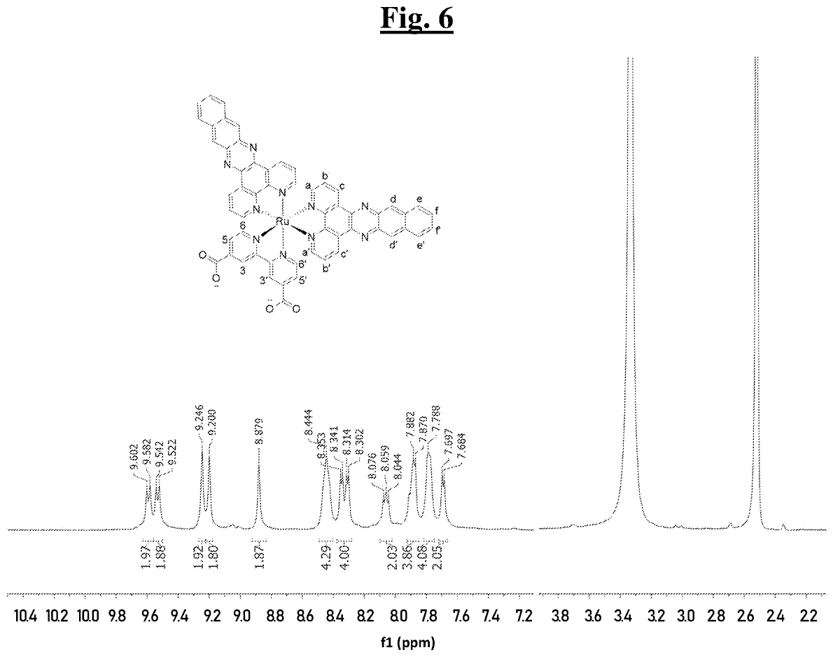

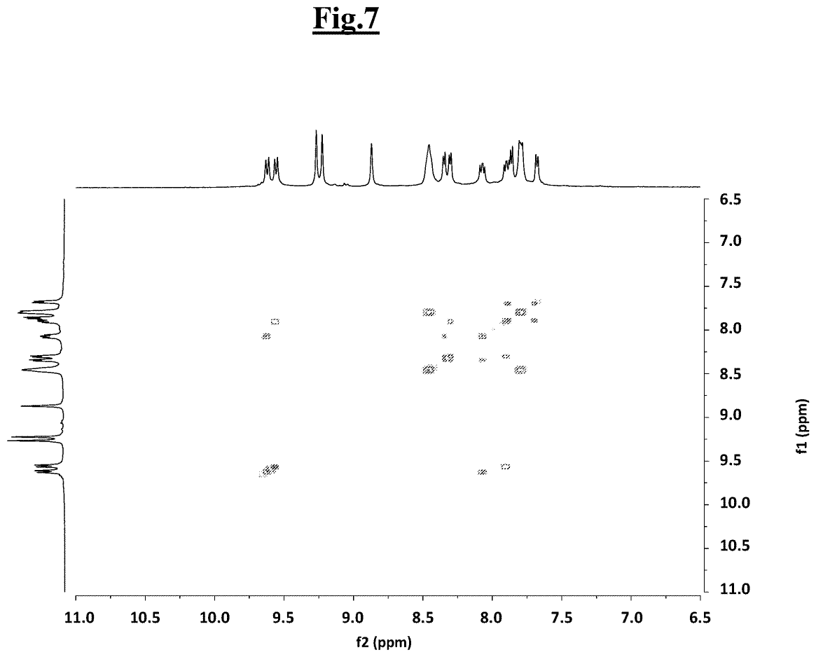

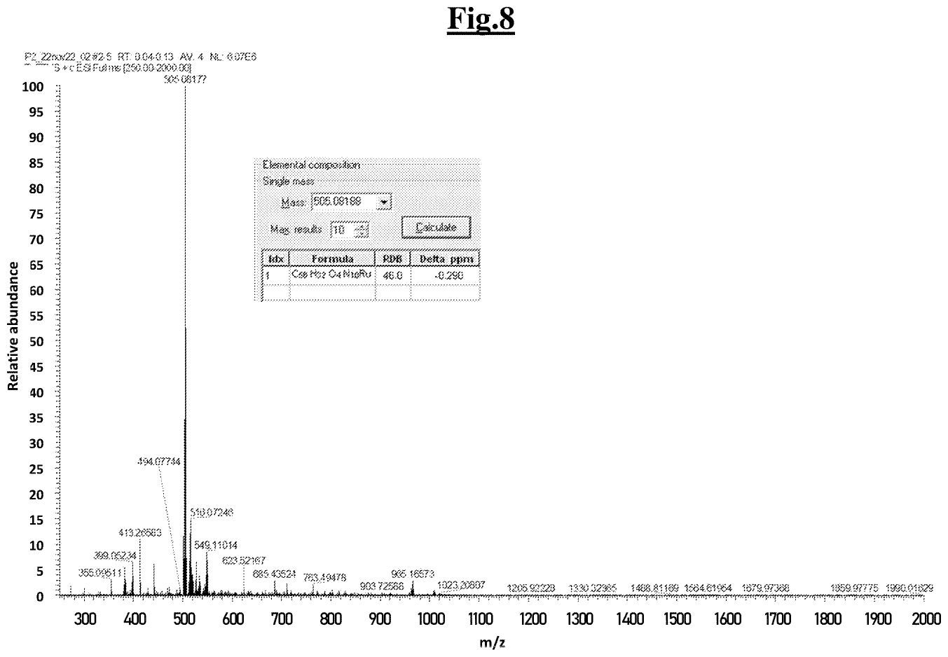

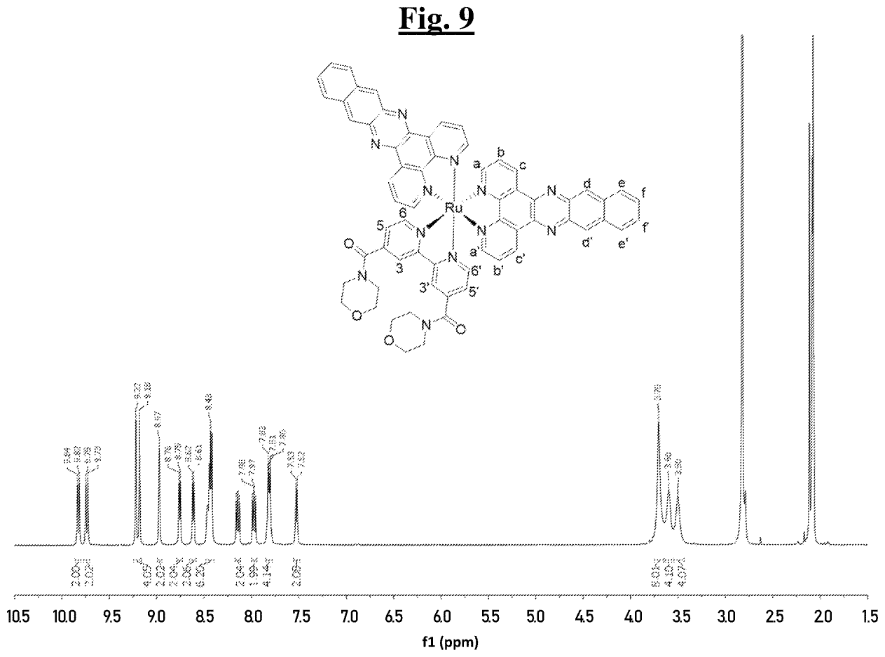

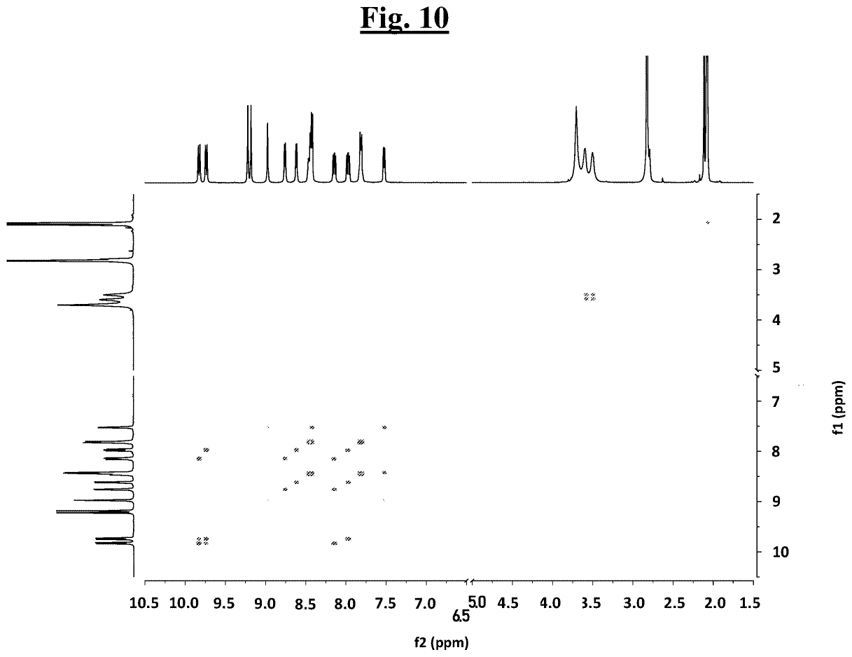

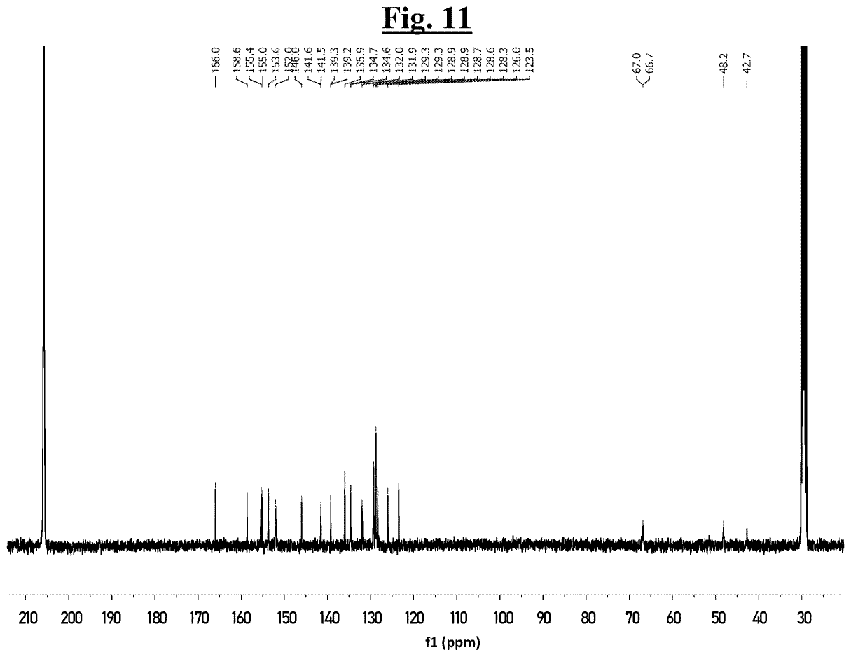

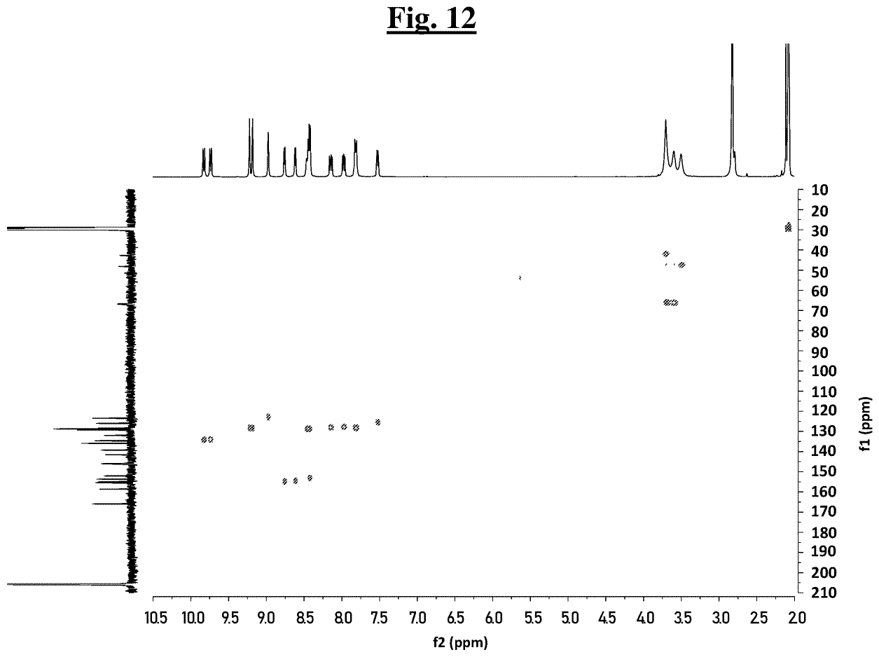

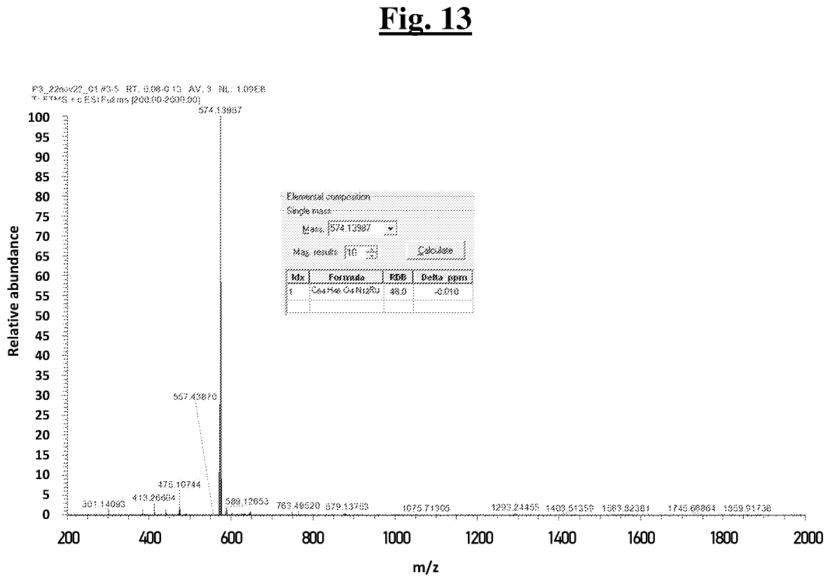

The NMR characterization data of the three complexes IIIa-IIIc synthesized as described above are shown below. The NMR characterization spectra of the three complexes are shown in FIGS. 1-13. The NMR spectra were recorded using a Bruker Advance 400 MHz.

### Complex IIIa

¹H-NMR (400 MHz, (CD₃)₂CO): δ 9.83 (d, J_{c-b}=8.0 Hz, 2H, Hc), 9.73 (d, J_{c′-b′}=8.0 Hz, 2H, Hc′), 9.23 (s, 2H, Hd), 9.20 (s, 2H, Hd′), 8.81 (s, 2H, H3/H3′), 8.67 (d, J=4.0 Hz, 2H, Ha), 8.62 (d, J_{a-b}=4.0, 2H, Ha′), 8.49-8.44 (m, 4H, He/He′), 8.18 (dd, J¹_{b-c}=8.0 Hz J²_{b-a}=4.0 Hz, 2H, Hb), 8.12 (d, J_{H2-H3}=4.0 Hz, 2H, H6/H6′), 7.95 (dd, J¹_{b′-c′}=8.0 Hz J²_{b′-a′}=4.0 Hz, 2H, Hb′), 7.87-7.80 (m, 4H, Hf/Hf′), 7.38 (d, J_{H3-H2}=4.0 Hz, 2H, H5/H5′), 2.61 (s, 6H, —CH₃) ppm.

¹³C-NMR (100 MHz, (CD₃)₂CO): δ 157.59, 155.08, 154.71, 152.34, 152.26, 152.13, 151.45, 141.51, 139.11, 135.80, 134.31, 134.23, 131.85, 131.77, 129.27, 129.19, 128.85, 128.80, 128.67, 128.45, 128.27, 125.89, 20.91 ppm.

HR-MS (ESI+) m/z: calcd. for C₅₆H₃₆N₁₀Ru [M-2PF₆⁻]²⁺475.10791 found: 475.10791.

Anal. calcd for C₅₆H₃₆F₁₂N₁₀P₂Ru: C, 54.24, N, 11.30, H, 2.93; found C, 54.00, N, 10.53, H, 3.24

### Complex IIIb

¹H-NMR (400 MHz, DMSO): δ 9.62 (d, J_{c-b}=8.0 Hz Hz, 2H, Hc), 9.56 (d, J_{c′-b′}=8.0 Hz, 2H, Hc′), 9.27 (s, 2H, Hd), 9.22 (s, 2H, Hd′), 8.87 (s, 2H, H3/H3′), 8.50-8.40 (m, 2H, Hf and Hf′), 8.35 (d, J_{a-b}=8.0 J_{a-b}=4.8 Hz, 2H, Ha), 8.31 (d, J_{a′-b′}=4.8 2H, Ha′) 8.08 (dd, 2H, Hb), 7.93-7.84 (m, 4H, Hb′ and H6/H6′), 7.82-7.75 (m, 4H, He and He′), 7.68 (d, J=5.2 Hz, 2H, H5/H5′) ppm.

The low solubility (0.5 mM in DMSO) prevented the recording of experiments ¹³C-NMR.

HR-MS (ESI+) m/z: calcd. for C₅₆H₃₂N₁₀O₄Ru [M-2PF₆⁻]²⁺ 505.08188, found: 505.08177.

Anal. calcd for C₅₆H₃₀N₁₀O₄Ru: C, 66.73, N, 13.90, H, 3.00; found C, 62.56, N, 12.62, H, 3.89.

### Complex IIIc

¹H-NMR (400 MHz, (CD₃)₂CO): δ 9.83 (d, J_{c-b}=8 Hz, 2H, Hc), 9.74 (d, J_{c′-b′}=8 Hz, 2H, Hc′), 9.22 (s, 2H, Hd), 9.18 (s, 2H, Hd′), 8.98 (s, 2H, H3/H3′), 8.76 (d, J_{a-b}=4 Hz, 2H, Ha), 8.62 (d, J_{a′-b′}=4 Hz, 2H, Ha′), 8.48-8.40 (m, 6H, H6/H6′, He/He′), 8.15 (dd, J¹_{b-c}=4 Hz, J²_{b-a}=8 Hz, 2H, Hb), 7.97 (dd, J¹_{b′-c′}=4 29 Hz, J²_{b′-a′}=8 Hz, 2H, Hb′), 7.85-7.79 (m, 4H, Hf/Hf′), 7.53 (d, J_{H3-H2}=4 Hz, 2H, H5/H5′), 3.71 (bs, 8H, —CH2 morpholine), 3.60 (bs, 4H, —CH2 morpholine), 3.50 (bs, 4H, —CH2 morpholine) ppm.

¹³C-NMR (100 MHz, (CD₃)₂CO): δ165.4; 157.9; 154.8; 154.4; 153.1; 151.4; 145.4; 140.9; 140.9; 138.6; 135.3; 134.1; 134.1; 131.4; 131.3; 128.7; 128.3; 128.2; 128.1; 128.1; 127.7; 125.4; 122.9 ppm.

HR MS (ESI+) m/z: calcd. for C₆₄H₄₆N₁₂O₄Ru [M-2PF₆⁻]²⁺ 574.13987, found: 574.13897.

Anal. calcd for C₆₄H₄₆N₁₂F₁₂O₄P₂Ru: C, 53.45, N, 11.69, H, 3.22; found C, 53.28, N, 11.04, H, 3.42.

### Characterisation of Synthesised Complexes: UV-Vis Absorption Profiles

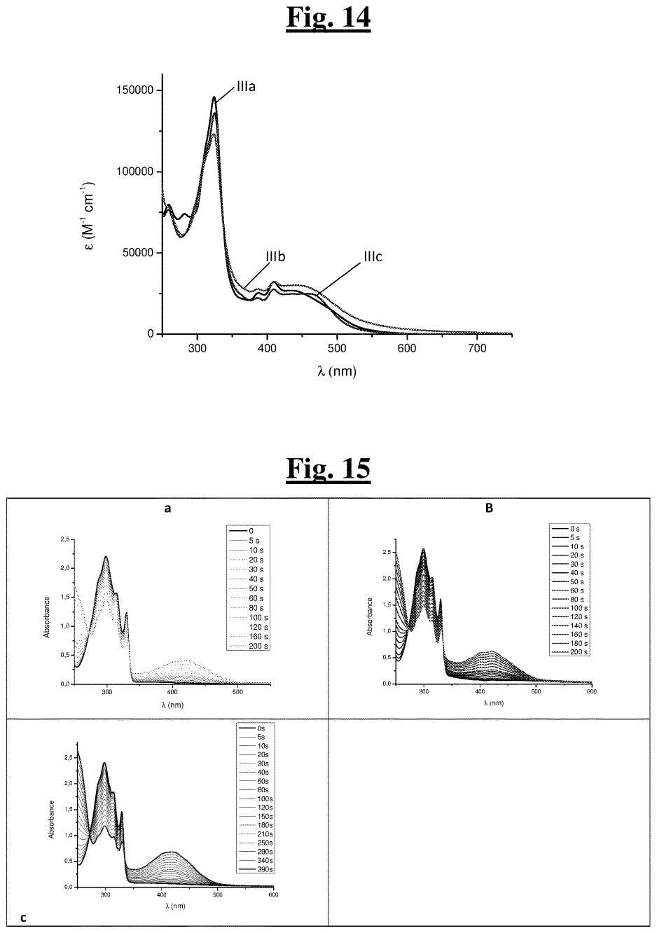

As shown in Table 1, the complexes of formula III show a broad ¹MLCT transition centred between 440 and 450 nm, and diagnostic ¹π-π* transitions of the aromatic part of the ligands centred between 408 and 320 nm in acetonitrile. FIG. 14 shows the absorption spectra of complexes IIIa-c in graphic form. All recorded absorption spectra shown in the present invention were recorded using a PerkinElmer Lambda 6 spectrophotometer.

### Determination of Singlet Oxygen Quantum Yield

The capacity of complexes IIIa-c to efficiently generate singlet oxygen (¹O₂) upon irradiation with light is a key factor in the evaluation of their potential as photosensitising agents for applications in photodynamic therapy. In this connection, the singlet oxygen quantum yields of complexes IIIa-c were determined by means of direct phosphorescence signal of ¹O₂ at 1270 nm generated by irradiation of acetonitrile solutions of the complexes and using dichlorotris(1,10-phenanthroline)ruthenium (II) chloride ([Ru(phen)₃Cl₂]) as a standard reference. A Horiba FluoroMax Plus spectrofluorometer was used for these measurements. The values are shown in Table 1.

TABLE 1
Chemical-physical characterization of ruthenium complexes
IIIa-c: absorption maxima, molar absorption coefficients
and singlet oxygen quantum yields obtained through (a)
direct phosphorescence signal measurements of ¹O₂ at 1270
nm and (b) indirect UV-visible measurements with DHN probe.

| | Solvent | | |
|---|---|---|---|
| | Acetonitrile | | |
| Com- | | Φ¹O₂ | |
| plex | λ_{abs}/nm (ε × 10M³⁻¹ cm)⁻¹ | (a) | (b) |
| IIIa | 440 (26.6) 409 (32.2) 387 (25.3) 324 (146.1) | 0.54 ± 0.06 | 0.44 |
| IIIb | 445 (30.0) 410 (32.4) 387 (27.8) 323 (123.2) | 0.50 ± 0.07 | 0.47 |
| IIIc | 455 (24.9) 408 (27.5) 384 (22.1) 325 (136.1) | 0.54 ± 0.6 | 0.42 |

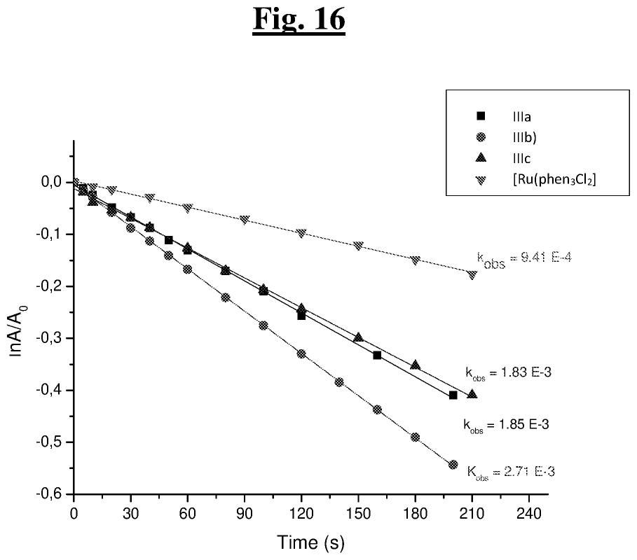

A confirmation of the production of singlet oxygen (¹O₂) with such a high quantum yields by the three IIIa-c molecular systems was obtained using 1,5-dihydroxynaphthalene (DHN) as an indirect chemical probe for ¹O₂ and [Ru(phen)₃]Cl₂ as a commercial standard. In the DHN assay, the probe is selectively and quantitatively oxidised in the presence of ¹O₂ to give the corresponding photo-oxidation product 5-hydroxy-1,4-naphthalenedione (Juglone). The production of ¹O₂ was assessed by monitoring the decrease in the DHN absorption band at λ=297 nm and the corresponding increase in the Juglone band at λ=427 nm. FIG. 15 shows the UV-Vis titrations obtained for IIIa-c in acetonitrile subjected to increasing irradiation times using an LED lamp with an emission spectrum between 400 and 500 nm. As shown in FIG. 15, irradiation of IIIa-c results in a strong decrease in the absorption of DHN, along with a simultaneous increase in the Juglone absorption band. The rate constants for the photo-oxidation process (k_{obs}) are obtained by plotting the semilogarithmic graph of ln(Aₜ/Aₒ) against the studied irradiation times, as shown in FIG. 16, in comparison with that found for [Ru(phen)₃]Cl₂ under the same experimental conditions. On this basis, the quantum yield of formation of ¹O₂ can be obtained via the equation

$${φ}_{Δ} = \frac{k {I}_{abs ST .}}{{k}_{ST .} {I}_{abs}} {φ}_{Δ ST ,}$$

where φ_{Δ} are the quantum yields of ¹O₂ production from the photosensitising agents, k are the rate constants obtained from the DHN assays, l_{abs} represents the absolute value for the integration of the absorption of the photosensitising agents in the spectral emission region of the LED lamp (400 nm to 500 nm), and the subscript ST indicates the commercial standard [Ru(phen)₃]Cl₂.

Considering that the standard quantum yield of ¹O₂ production by [Ru(phen)₃]Cl₂ is 0.38 and that the k_{obs} obtained are 1.85×10⁻³ for complex IIIa, 2.71×10⁻³ for complex IIIb, 1.83×10⁻³ for complex IIIc and 9.41×10⁻⁴ for the reference (FIG. 16), the quantum yields of ¹O₂ production for complexes IIIa-c are 0.44, 0.47 and 0.42, respectively, which are agreement with those obtained by direct measurement of the phosphorescence signal (Table 1).

Interaction with Calf Thymus DNA

The ability of complexes IIIa-c to bind DNA was assessed on calf thymus DNA (ct-DNA) monitoring the changes in the UV-Vis spectra of the 322 nm band upon titration of a solution of the complex at a fixed concentration (10 μM) in a buffer solution (Tris-HCl 10 mM, NaCl 50 mM, pH 7.2) with increasing amounts of biopolymer. At each addition, samples were incubated at room temperature during 5 minutes and UV-Vis spectra were recorded using a Tris-HCl buffered solution containing equal amounts of ct-DNA as a blank in order to eliminate the intrinsic contribution to absorbance of the ct-DNA itself. The binding constants found for complexes IIIa-c are shown in Table 2.

TABLE 2
Binding constants (K_{b}) of ruthenium complexes
synthesized in this invention with ct-DNA

| | Kb (ct- DNA) |
|---|---|
| IIIa | 7.49 × 105 |
| IIIb | 2.34 × 106 |
| IIIc | 8.75 × 105 |

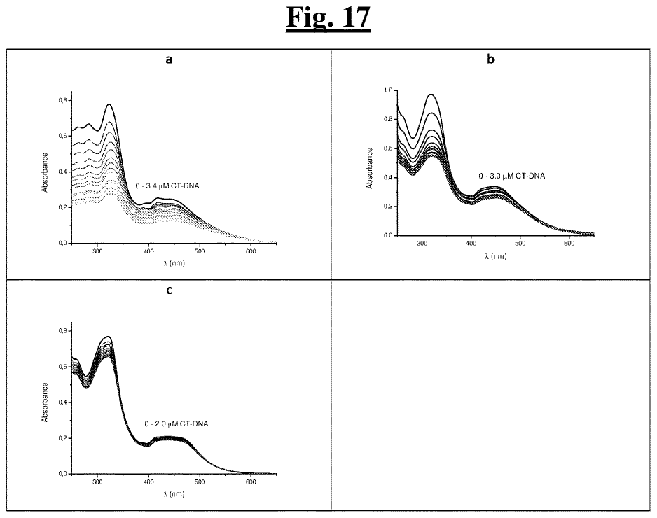

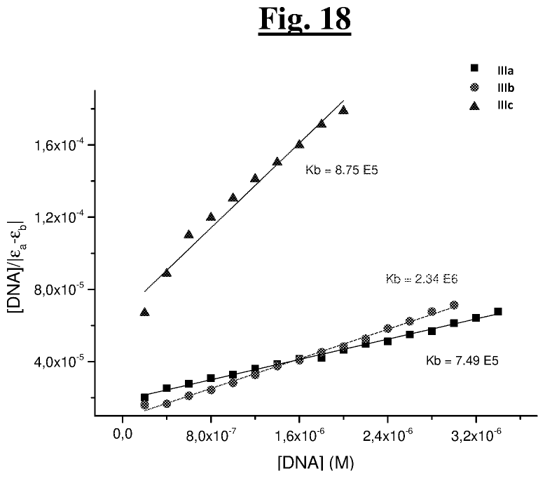

As shown in FIG. 17, the consecutive addition of ct-DNA (0 to 3.4 μM) results in a hypochromism of the whole absorption spectrum of complexes IIIa-c. The intrinsic binding constant K_{b} of complexes IIIa-c with ct-DNA was determined using the equation

$$\frac{[ DNA ]}{( {ε}_{a} - {ε}_{f} )} = \frac{[ DNA ]}{( {ε}_{b} - {ε}_{f} )} + \frac{1}{{K}_{b} ( {ε}_{b} - {ε}_{f} )}$$

where [DNA] is the concentration of ct-DNA in base pairs, and the apparent adsorption coefficients εₐ, ε_{f} and ε_{b} are A_{obs}/[Ru], the molar extinction coefficient for the unbound complex and the molar extinction coefficient for the Ru complexes in their fully DNA-bound form, respectively. The binding constants (K_{b}) are obtained as the ratio of the slope and the intercept of the linear regression of [DNA]/|εₐ-ε_{f}/plotted against [DNA], as shown in FIG. 18.

### Activity of Compound IIIa

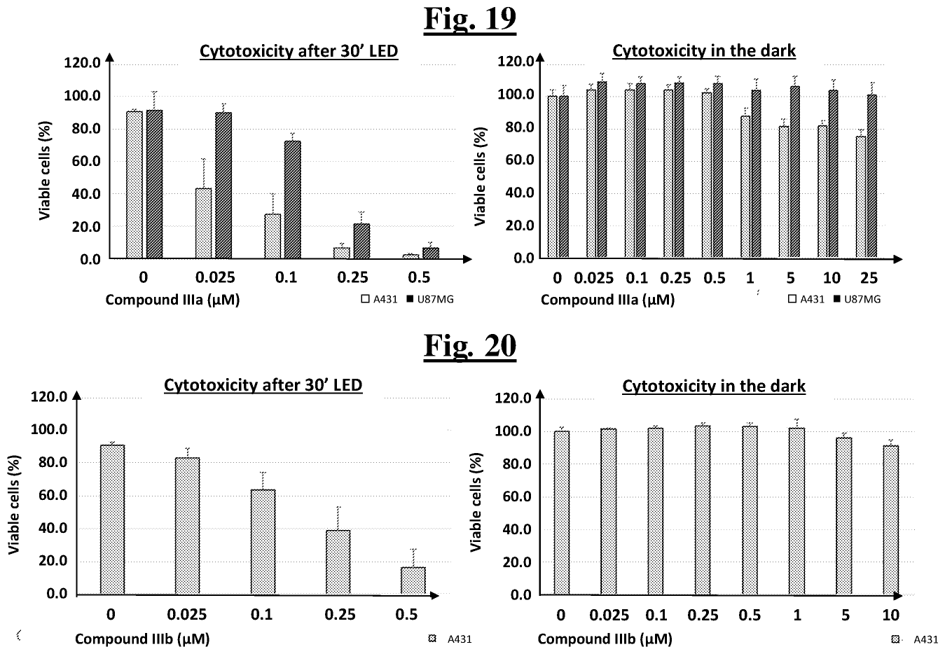

The activity of compound IIIa as a photosensitising agent in photodynamic therapy was evaluated on two in-vitro tumour models: squamous cell carcinoma of the epidermis (A431) and glioblastoma multiforme (U87MG). Cells cultured on 96-well plates were treated with increasing concentrations of IIIa dispersed in the culture medium for one hour. At the end of the incubation, the medium was replaced, and the cells were exposed to LED light (λₘₐₓ 462 nm) for 30 minutes, or were left in the dark during the same time. At the end of the 30 minutes, the cells were placed back and maintained in the incubator for 24 hours before measuring the metabolic activity by MTT assay. Six replicates were used for each condition, and cell viability of 100% was calculated from the response of untreated cells not exposed to LED light. On both models, compound IIIa showed high potency and specificity. In particular, compound IIIa induced extensive cell death after LED irradiation even at nanomolar concentrations, while exhibiting little or no toxicity when it was not activated by light, as shown in FIG. 19. The high specificity of IIIa, defined as toxicity only when exposed to light, is evident from the difference in IC₅₀ values, which indicate a potent cytotoxic activity in the presence of light irradiation and substantial inertia in the dark at the concentrations tested, as shown in Table 3. This indicates that compound IIIa has a great potential as a photosensitising agent in the photodynamic therapy for treating various types of tumours.

TABLE 3
Mean IC₅₀ values obtained from in-vitro cytotoxicity
experiments on compound IIIa tumour models

| | IC₅₀ (maximum response set to zero) | | |
|---|---|---|---|
| Cell cultures | | 30′ LED light exposure | In the dark |

| | | | |
|---|---|---|---|
| A431 | 0.0253 | μM | >40 μM |
| U87MG | 0.164 | μM | >40 μM |

### Activity of Compound IIIb

The activity of compound IIIb as a photosensitising agent in photodynamic therapy was evaluated on an in vitro tumour model, i.e., squamous cell carcinoma of the epidermis (A431), following the same protocol as compound IIIa. Compound IIIb showed high potency and specificity. In particular, IIIb induced extensive cell death after LED irradiation even at nanomolar concentrations, while exhibiting little or no toxicity when it was not activated by light, as shown in FIG. 20. The high specificity of IIIb, defined as toxicity only when exposed to light, is evident from the difference in IC₅₀ values, which indicate a potent cytotoxic activity in the presence of light irradiation and substantial inertia in the dark at test concentrations, see Table 4. This indicates that compound IIIb has a great potential as a photosensitising agent in the photodynamic therapy for treating various types of tumours.

TABLE 4
Mean IC₅₀ values obtained from in vitro cytotoxicity
experiments on compound IIIb tumour models

IC₅₀ (maximum response set to zero)

| Cell cultures | 30′ LED light exposure | In the dark |
|---|---|---|
| A431 | 0.191 μM | >40 μM |

### Formulation of Complex IIIb-Loaded Cubosomes: Chemical-Physical Characterisation

The formulation of cubosomes loaded with complex IIIb was prepared using monoolein, i.e. glycerol monooleate (1-monooleoylglycerol, RYLO MG 19 PHARMA, 98.1 wt. %, supplied by Danisco A/S), and Pluronic F108 (PEO132-PPO50-PEO132, purchased from Sigma Aldrich) as stabilising agent. Fresh distilled water purified with a Milli-Q system (Millipore) was used to prepare each sample and it was filtered through a 0.22 μm pore size hydrophilic filter before each sample preparation was used.

Cubosomes were prepared by melting the monoolein at 40° C. and dispersing the melted monoolein with the help of an ultrasonic bath. An appropriate amount of an aqueous solution of Pluronic F108 stabiliser was then added to the lipid phase, and the mixture was subjected to ultrasonication with a UP100H ultrasonic processor developed by Hiescher (90% amplitude; 1 s ON, 1 s OFF) for cycles of 5, 4, 3, 2 and 1 min.

The average hydrodynamic diameter and polydispersity index, as a measure of the width of the particle size distribution, were determined by Dynamic Light Scattering using a nano Zetasizer (Malvern Instrument). The samples were backscattered using a helium-neon laser (633 nm) at an angle of 173° and at a constant temperature of 25° C. The zeta-potential was estimated with the nano Zetasizer using the M3-PALS (Phase Analysis Light Scattering) technique.

The macroscopic appearance of the obtained sample was that of a fluid, opaque, pale orange aqueous dispersion, due to the presence of the Ruthenium complex. The investigation of the complex IIIb-loaded cubosomes started by assessing the encapsulation efficiency of the ruthenium complex. To this purpose, the complex IIIb-loaded cubosomes were separated from the free complex IIIb by dialyzing the formulation through a 14 kDa molecular weight cutoff tubular cellulose membrane (by Sigma Aldrich) against 2 L of water for 2 h, changing the water after one hour, at room temperature. Then, after disintegration of the formulation in methanol, in which all components are soluble, using a Synergy 4 multiplate reader (BioTek, Winooski, USA), the encapsulation/entrapment efficiency was evaluated by UV-Visible spectroscopy at 325 nm. Specifically, this efficiency was calculated by the expression:

$$Encapsulation efficiency = \frac{mass of drug after dialysis}{mass of weighted drug} \times 100 \%$$

The final concentration of IIIb was 0.02% (w/w) with an encapsulation efficiency value of 60±5%. Therefore, the final composition of complex IIIb-loaded cubosomes was
monoolein/Pluronic F108/complex IIIb/water=3.30/0.03/0.02/96.65% (w/w).

The samples were visually inspected before any measurement by Dynamic Light Scattering measurement to check the absence of aggregates or phase separation. Moreover, the colloidal system was characterised by nanoparticles with an average diameter of 142±1 nm, a polydispersity index of 0.13±0.01 and a zeta potential of −30±2 mV.

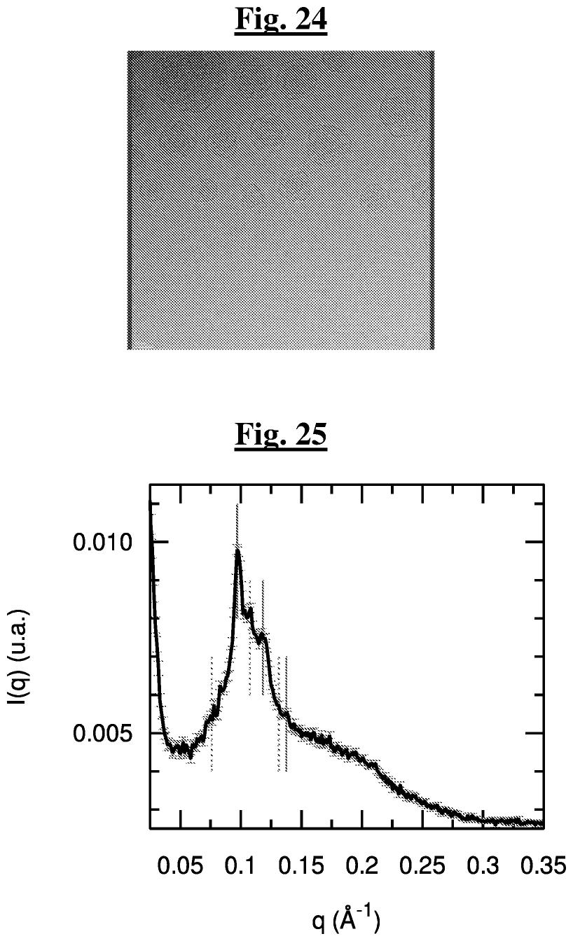

The morphology of IIIb-loaded cubosomes was revealed by transmission electron microscopy at cryogenic temperatures (Cryo-TEM). As shown in FIG. 24, the cubosomes appear as spherical nanoparticles of different sizes with an inner structure characterised by a dark matrix and alternating bright spots, representing the lipid bilayer and water channels, respectively.

The inner nanostructure of complex IIIb-loaded cubosomes was assessed by means of Small Angle X-ray Scattering (SAXS) experiments. In particular, the recorded SAXS diffractogram of complex IIIb-loaded cubosomes, shown in FIG. 25, strongly suggests the simultaneous presence of two bicontinuous cubic phases, Pn3m and Im3m, characterised by lattice parameters of 92±1 Å and 117±1 Å and water channel radii of 38±1 Å and 37±1 Å, respectively.

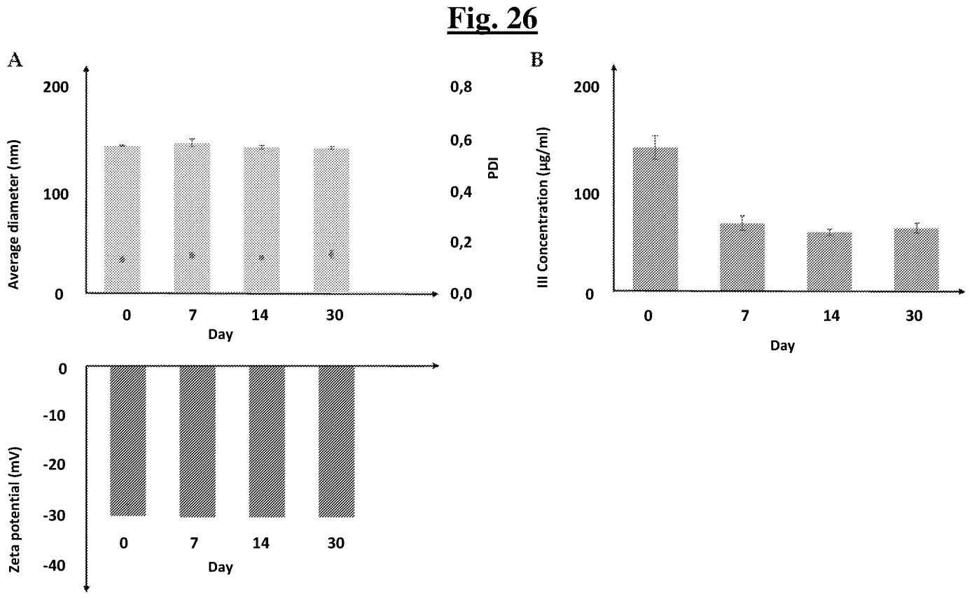

Information on the formulation stability was obtained by monitoring the mean diameter, the polydispersity index PDI and the zeta potential over a period of 30-day, as shown in FIG. 26A. Moreover, the chemical stability of complex IIIb encapsulated in the cubosomes was evaluated by analysing the formulation through UV-Visible spectroscopy, as shown in FIG. 26B. The study of the particle size distribution revealed an optimal stability of the formulation, as shown in FIG. 26.

Indeed, the mean diameter did not appreciably change during the 30-day storage at 25° C., showing a value around 140 nm throughout the study. The polydispersity index remained almost constant and below 0.15, which confirms t a retention of the fairly narrow size distribution during storage. Moreover, the value of the zeta potential did not change, with recorded values of ca. −30 mV. In addition, UV-Visible analysis revealed a decrease in the complex IIIb concentration between day 0 and day 7. However, the complex IIIb concentration remained unchanged from day 7 until the end of the investigation.

### Activity of Compound IIIb Encapsulated in Cubosomes

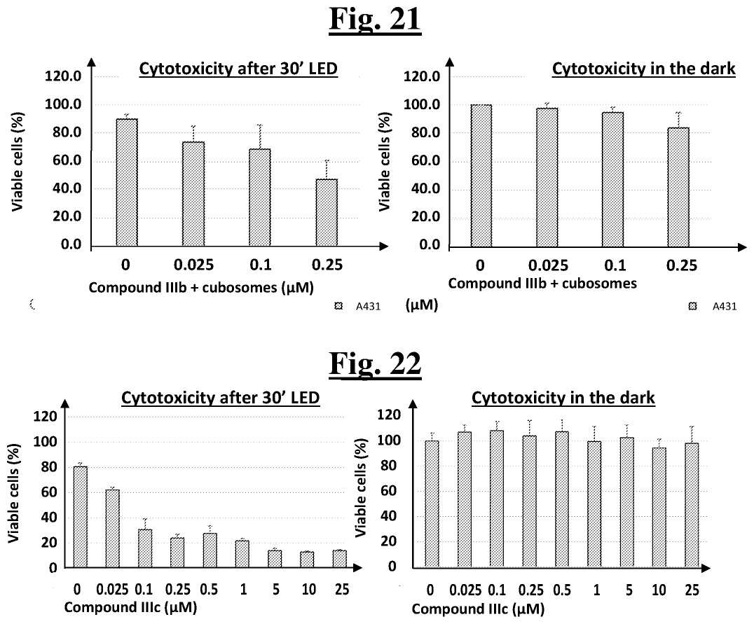

The activity of compound IIIb encapsulated in cubosomes as a photosensitising agent in photodynamic therapy was evaluated on an in vitro tumour model: squamous cell carcinoma of the epidermis (A431), following the same protocol as compound IIIa and IIIb as such. Compound IIIb encapsulated in cubosomes induced extensive cell death after LED irradiation even at nanomolar concentrations, while exhibiting little or no toxicity when it was not activated by light, as shown in FIG. 21. The encapsulation in cubosomes leads to a reduced IC₅₀ value, in comparison with IIIb as a free compound (Tables 4 and 5). However, the incorporation into a delivery system can bring numerous benefits, including increased distribution in the tumour after in vivo administration, increased stability and solubility, and improved crossing of biological barriers.

TABLE 5
Mean IC₅₀ values obtained from in vitro cytotoxicity experiments
on tumour models of compound IIIb encapsulated in cubosomes

IC₅₀ (maximum response set to zero)

| Cell cultures | 30′ LED light exposure | In the dark |
|---|---|---|
| A431 | 0.367 μM | ND |

Moreover, it should be noted that the medicament described above represents the first example of a Ru(II) polypyridyl complex encapsulated in cubosomes reported in the literature to date.

The foregoing description of embodiments of the invention, and examples thereof, are capable of showing the invention from a conceptual point of view in such a way that others, using the known technique, will be able to modify and/or adapt, in various applications, those embodiments without further research and without departing from the inventive concept, and, therefore, it is understood that such adaptations and modifications will be considered equivalent to the embodiments described. The means and materials for putting into practice the various functions may be of various kinds without departing from the scope of the invention. It is understood that the expressions or terminology used are purely descriptive and, therefore, not limiting.

### Activity of Compound IIIc

The activity of compound IIIc as a photosensitising agent in photodynamic therapy was evaluated on an in-vitro tumour model, Methastatic Lung Adenocarcinoma (Calu-3), following the same protocol as compounds IIIa and IIIb. Compound IIIc showed high potency and specificity. In particular, IIIc induced extensive cell death after LED irradiation even at nanomolar concentrations, while exhibiting little or no toxicity when it was not activated by light, as shown in FIG. 22. The high specificity of IIIc, defined as toxicity only when exposed to light, is evident from the difference in IC50 values, which indicate a potent cytotoxic activity in the presence of light irradiation and substantial inertia in the dark at test concentrations, see Table 6. This indicates that compound IIIc has a great potential as a photosensitising agent in the photodynamic therapy for treating various types of tumours.

TABLE 6
Mean IC50 values obtained from in-vitro cytotoxicity
experiments on compound IIIc tumour models

IC50 (maximum response set to zero)

| Cell cultures | 30′ LED light exposure | In the dark |
|---|---|---|
| Calu-3 | 0.0639 μM | >40 μM |

### Activity of Compound IIIc Encapsulated in Cubosomes

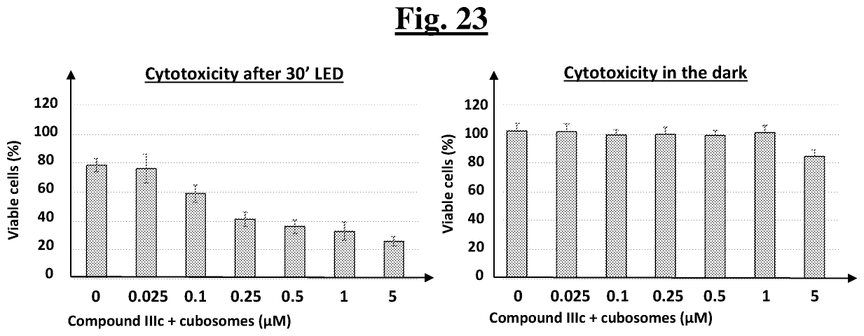

The activity of compound IIIc encapsulated in cubosomes as a photosensitising agent in photodynamic therapy was evaluated on an in vitro tumour model: Methastatic Lung Adenocarcinoma (Calu-3), following the same protocol as compound IIIa and IIIb. Compound IIIc encapsulated in cubosomes induced extensive cell death after LED irradiation even at nanomolar concentrations, while exhibiting little or no toxicity when it was not activated by light, as shown in FIG. 23. The encapsulation in cubosomes leads to a reduced IC50 value, in comparison with IIIc as a free compound (Tables 6 and 7). However, the incorporation into a delivery system can bring numerous benefits, including increased distribution in the tumour after in vivo administration, increased stability and solubility, and improved crossing of biological barriers.

TABLE 7
Mean IC50 values obtained from in vitro cytotoxicity experiments
on tumour models of compound IIIc encapsulated in cubosomes

IC50 (maximum response of zero)

| Cell cultures | 30′ LED light exposure | In the dark |
|---|---|---|
| Calu-3 | 0.144 μM | >5 μM |

The foregoing description of exemplary embodiments and specific examples of the invention will so fully reveal the invention according to the conceptual point of view, so that others, by applying current knowledge, will be able to modify and/or adapt such embodiments for various applications without further research and without parting from the invention, and, accordingly, it is to be understood that such adaptations and modifications will have to be considered as equivalent to the specific embodiment and to the examples. The means and the materials to perform the various functions described herein could have a different nature without, for this reason, departing from the scope of the invention. It is to be understood that the phraseology or terminology that is employed herein is for the purpose of description and not of limitation.

### BIBLIOGRAPHICAL REFERENCES

1) Wang, L. et al. “π-Expansive Heteroleptic Ruthenium (II) Complexes as Reverse Saturable Absorbers and Photosensitizers for Photodynamic Therapy” Inorg. Chem. 56, 3245-3259 (2017).
2) Turro et al., “Excited state dynamics of two new Ru(II) cyclometallated dyes: Relation to cells for solar energy conversion and comparison to conventional systems” J. Phys. Chem. C (2012).

1. Ruthenium (II) coordination compounds having formula: 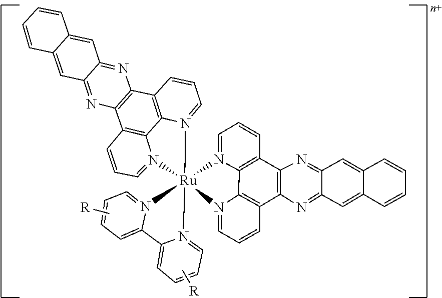
wherein n is selected between 0 and 2,
wherein R is a substituent group selected from the group consisting of:
a methyl group;
a phenyl group;
a —CH₂NH₂ group;
a —CH₂OH group;
a carboxyl group —COOH;
an ester group —COOR₁,
where R₁ is selected from the group consisting of:
a linear or branched alkyl group with a number of carbon atoms in the range from 1 to 4;
an unsubstituted benzyl group;
a polyethylene glycol methoxy group H(OCH₂CH₂)₁₋₄OH;
an amide group —CONR₂R₃,
where
R₂ is hydrogen and R₃ is a group selected from the group consisting of:
a linear or branched alkyl group with a number of carbon atoms comprised in the range from 1 to 4;
a —(CH₂)₂NH₂ group;
a phenyl group,
or
R₂ and R₃ are linear or branched alkyl groups with a number of carbon atoms in the range from 1 to 4,
or
NR₂R₃ is a group selected from the group consisting of:
a cycloalkyl secondary amine with a number of carbon atoms in the range from 4 to 5;
a heterocycloalkyl secondary amine comprising a heteroatom selected between nitrogen and oxygen,
for use as photosensitising agents in a photodynamic therapy for treating neoplasms.

2. The coordination compounds for use according to claim 1, wherein each substituent group R is bonded to a carbon atom at a position selected between the 5,5′ position and the 4,4′ position of each pyridine ring of the 2,2′-bipyridine group:

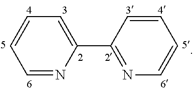 [SMILES: c1ccc(-c2ccccn2)nc1]

3. The coordination compounds for use according to claim 1, wherein said substituent groups R are bonded to carbon atoms in symmetrical 5,5′ or 4,4′ positions of respective pyridine rings of the 2,2′-bipyridine group

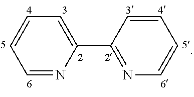 [SMILES: c1ccc(-c2ccccn2)nc1]

4. A photosensitizer medicament for photodynamic therapy to treat neoplasms comprising a ruthenium (II) coordination compound as a photosensitising agent, wherein said ruthenium (II) coordination compound has the formula: 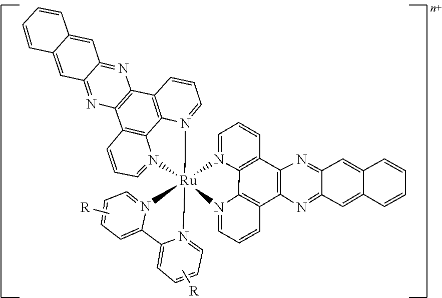
wherein n is selected between 0 and 2,
wherein R is a substituent group selected from the group consisting of:
hydrogen;
a methyl group;
a phenyl group;
a —CH₂NH₂ group;
a —CH₂OH group;
a carboxyl group —COOH;
an ester group —COOR₁,
where R₁ is selected from the group consisting of:
a linear or branched alkyl group with a number of carbon atoms in the range from 1 to 4;
an unsubstituted benzyl group;
a polyethylene glycol methoxy group H(OCH₂ CH₂)₁₋₄OH;
an amide group —CONR₂R₃,
where
R₂ is hydrogen and R₃ is a group selected from the group consisting of:
a linear or branched alkyl group with a number of carbon atoms comprised in the range from 1 to 4;
a —(CH₂)₂NH₂ group;
a phenyl group,
or
R₂ and R₃ are linear or branched alkyl groups with a number of carbon atoms in the range from 1 to 4,
or
NR₂R₃ is a group selected from the group consisting of:
a cycloalkyl secondary amine with a number of carbon atoms in the range from 4 to 5;
a heterocycloalkyl secondary amine comprising a heteroatom selected between nitrogen and oxygen,
wherein said coordination compound is incorporated in liquid-crystalline lamellar and/or non-lamellar lipid nanoparticles.

5. The medicament for photodynamic therapy according to claim 4, wherein said nanoparticles are cubosomes.

6. The medicament for photodynamic therapy according to claim 4, wherein said nanoparticles are selected from the group consisting of:
hexosomes;
ethosomes;
transferosomes;
solid lipid nanoparticles;
nanoemulsions;
a combination of the above.

7. The medicament for photodynamic therapy according to claim 4, wherein said nanoparticles are liposomes.

8. A process for obtaining a ruthenium (II) coordination compounds having formula: 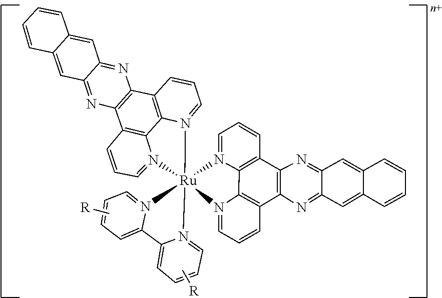
where n is selected between 0 and 2,
where R is a substituent group selected from the group consisting of:
hydrogen;
a methyl group;
a phenyl group;
a —CH₂NH₂ group;
a —CH₂OH group;
a carboxyl group —COOH;
an ester group —COOR₁,
where R₁ is selected from the group consisting of:
a linear or branched alkyl group with a number of carbon atoms in the range from 1 to 4;
an unsubstituted benzyl group;
a polyethylene glycol methoxy group having the formula H(OCH₂CH₂)₁₋₄OH;
an amide group —CONR₂R₃,
where
R₂ is hydrogen and R₃ is a group selected from the group consisting of:
a linear or branched alkyl group with a number of carbon atoms in the range from 1 to 4;
a —(CH₂)₂NH₂;
a phenyl group,
or
R₂ and R₃ are linear or branched alkyl groups with a number of carbon atoms in the range from 1 to 4,
or
NR₂R₃ is a group selected from the group consisting of:
a cycloalkyl secondary amine with a number of carbon atoms in the range from 4 to 5;
a heterocycloalkyl secondary amine comprising a heteroatom selected between nitrogen and oxygen,
said process comprising the sequence of steps of:
obtaining a reactive polymeric precursor [Ru(CO)₂Cl₂]ₘ by reaction between ruthenium (Ill) chloride and paraformaldehyde;
obtaining an intermediate complex trans-Cl[Ru(NN)Cl₂(CO)₂]: 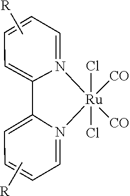
where NN is a 2,2′-bipyridine ligand symmetrically functionalised with two equal functional R groups,
by reaction of said reactive polymer precursor [Ru(CO)₂Cl₂]ₘ with a predetermined symmetrically functionalized 2,2′-bipyridine group with said two equal R functional groups,
obtaining said ruthenium (II) coordination compounds by reaction of said intermediate complexes 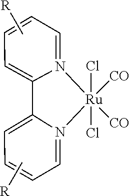
with benzo[i]dipyridine[3,2-a:2′,3′-c]phenazine.

9. The process according to claim 8, wherein each substituent group R is bonded to a carbon atom at a position selected between the 5,5′ position and the 4,4′ position of each pyridine ring of the 2,2′-bipyridine group

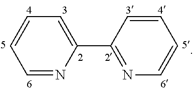 [SMILES: c1ccc(-c2ccccn2)nc1]

10. The process according to claim 8, wherein said substituent groups R are bonded to carbon atoms in symmetrical 5,5′ or 4,4′ positions of the respective pyridine rings of the 2,2′-bipyridine group 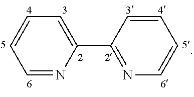 [SMILES: c1ccc(-c2ccccn2)nc1]
